(19) United States
(12) Patent Application Publication
(10) Pub. No.: US 2007/0219498 A1
(43) Pub. Date: Sep. 20, 2007

(54) SHOCK ABSORBER FOR AUTOMATIC INJECTOR

(75) Inventors: Greg Malone, Encinitas, CA (US); Eric Warner, Oceanside, CA (US)

Correspondence Address: WILSON SONSINI GOODRICH & ROSATI, 650 PAGE MILL ROAD, PALO ALTO, CA, 94304-1050, US

(73) Assignee: Verus Pharmaceuticals, Inc., San Diego, CA (US)

(21) Appl. No.: 11/680,374

(22) Filed: Feb. 28, 2007

Related U.S. Application Data
(60) Provisional application No. 60/743,379, filed on Feb. 28, 2006.

(51) Int. Cl. A61M 5/20

(52) U.S. Cl. 604/135

(57) ABSTRACT

An automatic injector comprising an improved energy management system as well as an improved shock absorber system adapted to reduce dynamic stresses on internal device components is provided. In particular, a shock absorber system comprising a stationary shock absorber is provided. In some embodiments, the stationary shock absorber is located in the nose of the automatic injector.

### PRIORITY CLAIM AND CROSS-REFERENCE TO RELATED APPLICATIONS

This application claims the benefit under 35 U.S.C. § 119(e) of U.S. Provisional Application No. 60/743,379, filed Feb. 28, 2006, which is incorporated herein by reference in its entirety.

### FIELD OF THE INVENTION

The present invention relates to the field of automatic injectors. In particular, the invention relates to an improved energy management system, as well as an improved shock absorber configuration as part of an improved shock absorber system for use in automatic injectors.

### BACKGROUND OF THE INVENTION

Automatic injectors have been used for self-administration of adrenaline (epinephrine), antihistamines and atropine. Automatic injectors have also been used by health care professionals for rapid and accurate subcutaneous or intramuscular injection of various medicaments. Thus, automatic injectors provide fast and convenient dosing for a variety of medicaments.

FIGS. 1-3 depict a prior art dual-use automatic injector 100. FIG. 1 is a side cutaway view of the automatic injector 100. FIG. 2 is a muzzle end-on view of the automatic injector 100; and FIG. 3 is a butt end-on view of the automatic injector 100. FIG. 7 depicts a syringe assembly 192, which has been removed from an automatic injector 100, and which may be used to manually deliver a dose of medicament to a patient.

The automatic injector 100 comprises a barrel 118 and a firing sleeve 122, which together form housing 188. The barrel 118 fits within the lumen of the firing sleeve 122; and the firing sleeve 122 is capable of sliding outside the barrel 118. For purposes of orientation, the automatic injector 100 can be envisioned as having a muzzle end 140 and a butt end 138. In the following description, “muzzle” or “forward” may be used as a modifier for any part, indicating a relative orientation toward the muzzle end 140, while the terms “butt” or “rear” may be used as a modifier for any part, indicating an orientation toward the butt end 138 of the automatic injector 100. Hereafter, the muzzle end may also be referred to a the front and the butt end may be referred to as the back or rear. Thus, where a first component that is located closer to the front end of the automatic injector 100 than a second component, the first component may be said to be “in front of” the second component. In some cases, the term “down” may indicate motion of a component forward during firing.

A trigger hole 142 in the butt of the barrel 118 lines up with a release aperture 172 in the butt of the firing sleeve 122, the operation of which will be described hereafter. A cylindrical spring guide 116 fits within the lumen of the barrel 118. Within the lumen of the spring guide 116 is a firing spring 114. A spring release 112 fits within the firing spring 114 in such a way as to hold the firing spring 114 in place in a cocked position. The spring release 112 has a spring release head 146, and a plurality of spring release legs 170, each of which terminates in a spring release hook 148. The muzzle end of the firing spring 114 abuts the rear of the spring release head 146. When the firing spring 114 is sufficiently compressed, the spring release legs 170 protrude through the firing spring 114, the firing bushing 120 and the trigger hole 142. The spring release legs 170 expand outward so that the spring release hooks 170 hold the spring release 112 in place, which in turn holds the firing spring 114 in a compressed (cocked) position until the automatic injector 100 is fired. The spring release hooks 148 are so shaped, and the trigger hole 142 and release aperture 172 are of such diameter, that when the firing sleeve 122 is moved forward relative to the barrel 118 of the automatic injector 100 with sufficient force, the inner walls of the release aperture 172 push spring release hooks 148 toward one another until they are capable of passing through the trigger hole 142. This releases the firing spring 114, allowing it to impart energy to other internal components of the automatic injector 100 as discussed in more detail below.

As depicted in FIG. 1, the firing spring 114 is in the above-described cocked position. A safety cap 124, having a stem 144, fits on the butt end 138 of the firing sleeve 122. The stem 144 of the safety cap 124 fits through the release aperture 172, between the release hooks 148 and through the trigger hole 142, thereby preventing the inward motion of the release hooks 148, thereby preventing firing of the automatic injector 100. Once the safety cap 124 is removed, however, the spring release hooks 148 are free to move toward one another. Motion of the firing sleeve 122 forward (relative to the barrel 118) with sufficient force will then cause the inner wall of the release aperture 172 to contact the outer edges of the spring release hooks 148, pushing them inward until they are free to pass through the trigger hole 142, thereby releasing the firing spring 114.

The automatic injector 100 also comprises a syringe assembly 192, which is also depicted in FIG. 7 without the other automatic injector components. The syringe assembly 192 comprises a syringe body 154, a plunger subassembly 196 and a needle hub subassembly 194. The syringe body 154 contains the medicament 102 and receives the plunger 152 of the plunger subassembly 196 in one end and is capped by a septum 156 and cap 168 on the other end, on which the needle hub subassembly 194 is seated.

The plunger subassembly 196 comprises the aforementioned plunger 152 for pushing liquid medicament 102 through and out of the syringe body 154 and into and through the needle hub subassembly 194. The plunger 152 is connected to a drive rod 104, which is connected to an adjustment screw 150 hi the depicted embodiment, the adjustment screw 150 fits within the drive rod 104 by means of screw threads 106, which allow the length between the butt end of the adjustment screw 150 and the muzzle end of the plunger 152 to be adjusted by turning the adjustment screw 150, if desired. In alternative embodiments, the adjustment screw 150 and drive rod 104 may be formed as an integral (non-adjustable) unit. Also depicted is a stop collar 110, which stops forward motion of the plunger subassembly 196. Thus, the stop collar 110 allows a portion of the medicament 102 to be retained in the syringe assembly 192 after automatic injection for use in an optional separate, manually administered dose. A bushing 108 encircles the drive rod 104 and ensures smooth movement of the drive rod 104 into the butt end of the syringe body 154.

The syringe body 154 has a rubber septum 156 covered with a cap 168, which retains the medicament 102 in a sealed environment until such time as an engagement needle 160 pierces the rubber septum 156. through a hole (not shown) in the cap 168.

A needle hub subassembly 194 fits on the muzzle end of the syringe body 154, specifically over the cap 168. The needle hub subassembly 194 comprises the aforementioned injection needle 164 for insertion into the body of a patient and delivery of medicament 102 into the body of the patient. The injection needle 164 is of a suitable gauge for subcutaneous and/or intramuscular injection, for example from 16 to 28 ga., particularly from 18 to 26 ga., more particularly from 20 to 26 ga., especially 21, 22, 23, 24 or 25 ga. Needle hub subassembly 194 also has a hub 158 that fits over the cap 168. The hub 158 forms the base to which the hub body 162 is attached. An engagement needle 160 protrudes from the within the hub body 162 and into the hub 158, directed toward the septum 156 through a hole (not shown) in the cap 168. The engagement needle 160 is adapted to penetrate the septum 156 through the hole (not shown) in the cap 168 when the syringe body 154 is pushed forward by the action of the firing spring 114 acting through the spring release head 146, the adjustment screw 150, the plunger 152 and the syringe body 154. The needle hub subassembly 194 further comprises a hub nose 176 through which passes the injection needle 164. The injection needle 164 is in fluid communication with the engagement needle 160. In some embodiments, the injection needle 164 and the engagement needle 160 are formed from a single tube that is sharpened at both ends. The hub body 162 has hub fins 174, which connect to the hub body 162 and the hub 158. Encircling the hub fins 174 and in contact with the hub 158 there is a shock absorber 134, which is a ring of elastic polymer material capable of absorbing and dispersing dynamic forces caused by the sudden stopping of the syringe assembly 192. In contact with the shock absorber 134 and also encircling the hub fins 174 is a shock absorber modifier 136, which is formed of a hard substance (e.g. metal) and serves to spread force of contact of a needle penetration controller 126 over the surface of the shock absorber 134.

The barrel 118 has a removable nose cap 128 fitted over the muzzle end thereof. The nose cap 128 is threaded, i.e. it has threads 184. Within the nose cap 128 there is a needle penetration controller 126. The needle penetration controller 126 is a relatively hard cylindrical structure that contacts the inner portion of the nose cap 128. During firing, the syringe assembly 192 moves down the barrel 118 (i.e. forward) until it impacts the needle penetration controller through the shock absorber 134 and shock absorber modifier 136, which causes the syringe assembly 192 to stop moving forward. When the syringe assembly 192 stops moving, the cap 168 is pushed forward until seats within the hub 158, while the engagement needle 160 pierces the septum 156 through the hole in the cap 168. Thus, the engagement needle 160 comes into contact with the medicament 102. The force of the firing spring 114 acting through the adjustment screw 150 and drive rod 104 then moves the plunger 152 forward, thereby expelling medicament 102 into and through the engagement needle 160, through and out of the injection needle 164 and into the body of the patient.

A return spring 132 provides dampening force in opposition to the forward motion of the syringe assembly 192. In the depicted embodiment, the return spring 132 is of smaller radius than the inside of the needle penetration controller 126, and extends from the inside surface of the nose cap 128 to the edge of the shock absorber modifier 136.

Prior to use, a needle sheath 166 fits through needle aperture 178 and over the end of injection needle 114 to protect the injection needle 164 from damage and to protect the user from accidental needle sticks. A sheath remover 130 fits over the end of the nose cap 128 and has a sheath receiving orifice 182 (FIG. 2), which is encircled by a plurality of projections 180 (FIG. 2), which securely engage the needle sheath 166 and allow the sheath 166 to be easily removed from the injection needle 164, by pulling forward on the sheath remover 130, with reduced risk of an accidental needle stick to the user.

The prior art automatic injector described above is similar in operation to embodiments described in U.S. Ser. No. 11/006,382, filed on Dec. 6, 2004, the contents of which is incorporated herein by reference in its entirety. Another prior art device is described in U.S. Pat. No. 4,031,893, the entire contents of which are incorporated herein by reference. (In particular FIG. 1 and the description thereof in U.S. Pat. No. 4,031,893 are expressly incorporated herein.)

Thus, “firing” the automatic injector means triggering release of the firing spring 114, injection of the medicament 102 into the patient, and all the intermediate steps carried out by the internal components of the automatic injector 100. Such intermediate steps include: (1) movement of the spring release 112 into contact with the adjustment screw 150, (2) movement of the syringe assembly 192 down the lumen of the barrel 118 until the injection needle 164 protrudes from the injection needle aperture 178 and into the patient's body; (3) impact of hub 158 with the needle penetration controller 126 through the shock absorber 134 and shock absorber modifier 136, thereby stopping the forward progress of the hub 158; (4) continued forward movement of the cap 168 forward until the engagement needle 160 pierces the septum 156 through the hole in the cap 168, thereby bringing the engagement needle 160 in fluid contact with the medicament 102; and (5) depression of the plunger subassembly 196 to push medicament 102 through the engagement needle 160 and out the end of the injection needle 164 and ultimately into the patient. Each of the steps delineated above entails the conversion of the potential energy of the compressed firing spring 114 into kinetic energy, which is expended in carrying out each step. Thus, it is necessary to use a firing spring 114 that is capable of storing (when compressed) and delivering (when released) a sufficient energy to sequentially execute each of these steps.

The depicted prior art automatic injector 100 is a dual-use automatic injector, supporting administration of both an automatically administered dose and a manually administered dose. After firing of the automatic injector 100, the nose cap 128 may be removed from the end of the barrel 118 by unscrewing it from the barrel 118. The syringe assembly 192 may then be removed from the automatic injector 100. A second, manual dose may be delivered by first removing the stop collar 110, thereby allowing further depression of the plunger assembly 196 through the adjustment screw 150. The injection needle is inserted into the patient's body and the plunger assembly 196 is depressed to inject the medicament 102 into the patient. Although the prior art device is depicted with a single stop collar 110, it is to be understood that the syringe assembly 192 may be manufactured with multiple stop collars, e.g. 2, 3 or more stop collars, which permit administration of 2, 3 or more additional, manual doses of the medicament.

In order to ensure that an automatic injection device will operate under a range of operating conditions, it is considered advantageous to use a firing spring that is capable of storing and delivering energy in excess of the minimum necessary to operate the device under normal conditions, e.g. room temperature. However, dynamic stresses placed on internal components of the automatic injector during firing limit the amount of energy that may be stored in, and delivered by, the firing spring in the prior art automatic injector.

There is thus a need for an automatic injector that has improved energy management features as compared to a prior art automatic injector. In particular, there is a need for an automatic injector capable of managing dynamic spring forces during firing, thereby reducing dynamic stresses on internal components of the automatic injector.

There is also a need for an automatic injector that is capable of taking advantage of a stronger spring.

There is also a need for an automatic injector that is capable of operating under a wider range of operating conditions, such as temperatures.

There is also a need for a multi-use injector that is capable of managing a firing spring having greater spring strength than the prior art injector.

There is further a need for a multi-use injector having a firing spring having greater usable potential energy stored therein than the prior art injector.

There is further a need for an automatic injector, especially a multi-use automatic injector, capable of operating under a wider range of operating conditions (such as temperatures) than the prior art automatic injector.

There is moreover a need for an automatic injector, especially a multi-use injector, that is easier to assemble than the prior art injector. In particular, there is a need for an automatic injector, especially a multi-use injector, that does not require placement of a shock absorber on the syringe assembly.

### SUMMARY OF THE INVENTION

The foregoing and further needs are met by embodiments of the invention, which provide an automatic injector device comprising a housing, a firing spring within the housing, a syringe assembly in front of the firing spring, a shock absorber system in front of the syringe assembly, and a trigger mechanism. The trigger mechanism is adapted to hold the firing spring in a cocked position until a user triggers release of the firing spring, thereby firing the automatic injector. The shock absorber system comprises a stationary shock absorber.

Additional needs are met by embodiments of the invention, which provide a single- or multi-use injector, comprising a housing, a firing spring within the housing, a syringe assembly in front of the firing spring, a shock absorber system, a trigger mechanism, and a means for removing the syringe assembly from the housing after the automatic injector has been fired. The shock absorber system comprises a stationary shock absorber. The trigger mechanism is adapted to hold the firing spring in a cocked position until a user fires the automatic injector.

Further needs are met by embodiments of the invention, which provide an automatic injector device comprising a housing, a firing spring adapted to release greater than about 12 lbs·in of kinetic energy and located within the housing, a syringe assembly in front of the firing spring, a shock absorber capable of managing energy imparted by a released firing spring of greater than about 12 lbs·in, and a trigger mechanism. The trigger mechanism is adapted to hold the firing spring in a cocked position until a user releases the firing spring, thereby firing the automatic injector.

Additional needs are met by embodiments of the invention, which provide an automatic injector device comprising a housing, a firing spring adapted to release less than about 8 lbs·in of kinetic energy and located within the housing, a syringe assembly in front of the firing spring, a shock absorber capable of managing energy imparted by a released firing spring of less than about 1 lbs·in, and a trigger mechanism. The trigger mechanism is adapted to hold the firing spring in a cocked position until a user releases the firing spring, thereby firing the automatic injector.

Further needs are met by embodiments of the invention, which provide an automatic injector device comprising a housing, a firing spring adapted to release about 8.7 lbs·in to about 12.3 lbs·in of kinetic energy and located within the housing, a syringe assembly in front of the firing spring, a shock absorber capable of managing energy imparted by a released firing spring of about 8.7 lbs·in to about 12.3 lbs·in, and a trigger mechanism. The trigger mechanism is adapted to hold the firing spring in a cocked position until a user releases the firing spring, thereby firing the automatic injector.

Further needs are met by embodiments of the invention, which provide an automatic injector device comprising a housing, a firing spring adapted to release about 8 lbs·in to about 10 lbs·in of kinetic energy and located within the housing, a syringe assembly in front of the firing spring, a shock absorber capable of managing energy imparted by a released firing spring of about 8 lbs·in to about 10 lbs·in, and a trigger mechanism. The trigger mechanism is adapted to hold the firing spring in a cocked position until a user releases the firing spring, thereby firing the automatic injector.

Further needs are met by embodiments of the invention, which provide an automatic injector device comprising a housing, a firing spring adapted to release about 9 lbs·in to about 12 lbs·in of kinetic energy and located within the housing, a syringe assembly in front of the firing spring, a shock absorber capable of managing energy imparted by a released firing spring of about 9 lbs·in to about 12 lbs·in, and a trigger mechanism. The trigger mechanism is adapted to hold the firing spring in a cocked position until a user releases the firing spring, thereby firing the automatic injector.

Still further needs are met by embodiments of the invention, which provide methods of reducing dynamic stresses on internal components of an automatic injector. The methods comprise providing the automatic injector with a shock absorber capable of managing energy released by a firing spring during firing.

Additional needs are met by embodiments of the invention, which provide an automatic injector device comprising a housing, a firing spring adapted to release less than about 29 lbs·in of energy and located within the housing, a syringe assembly in front of the firing spring, and a trigger mechanism. The trigger mechanism is adapted to hold the firing spring in a cocked position until a user releases the firing spring, thereby firing the automatic injector.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a cutaway side view of a prior art automatic injector.

FIG. 2 shows a muzzle end-on view of the prior art automatic injector.

FIG. 3 shows a butt end-on view of the prior art automatic injector

FIG. 4 shows a cutaway side view of an automatic injector of the invention, comprising the improved shock absorber system of some embodiments of the invention.

FIG. 5 shows a muzzle end-on view of the automatic injector of the invention, including the improved shock absorber system of some embodiments of the invention.

FIG. 6 shows a butt end-on view of the automatic injector of and embodiment of the invention.

FIG. 7 depicts a syringe assembly according to some embodiments of the invention that has been removed from an automatic injector of some embodiments of the invention.

FIG. 8 shows an expanded view of the muzzle end of an embodiment of the automatic injector of the invention, including an improved shock absorber, a needle penetration controller a the return spring.

FIG. 9 is a flow chart depicting the release of energy during operation of the automatic injector of the invention.

### DETAILED DESCRIPTION OF THE INVENTION

The present invention provides an improved energy management system and automatic injector. The improved automatic injector comprises energy management features that reduce dynamic stresses on internal components during actuation (firing) of the device. The details of such improved energy management features are discussed in detail below. In some embodiments, the energy management features include a stationary shock absorber, especially a stationary shock absorber located in the nose of the automatic injector.

In some embodiments, the invention provides an automatic injector device comprising: (a) a housing; (b) a firing spring within the housing; (c) a syringe assembly in front of the firing spring; (d) a shock absorber system in front of the syringe assembly, wherein the shock absorber system comprises a stationary shock absorber; and (e) a trigger mechanism, wherein the trigger mechanism is adapted to hold the firing spring in a cocked position until a user triggers release of the firing spring, thereby firing the automatic injector. In some embodiments, the housing has a nose cap and the stationary shock absorber is located within the nose cap. In some embodiments, the nose cap is removable. In some embodiments, the nose cap is threaded and removable by twisting the nose cap about an axis of the nose cap. In some embodiments, twisting the nose cap after the automatic injector has been fired causes the firing spring to become fully unloaded or substantially fully unloaded before the nose cap is removed. In some embodiments, the nose cap has an inner shelf and the shock absorber rests (at least in part) on the inner shelf.

In some embodiments, the shock absorber system further comprises a needle penetration controller located between the syringe assembly and the shock absorber. In some embodiments, the shock absorber system does not comprise a shock absorber on the syringe assembly; in other embodiments, the shock absorber system does include a shock absorber on the syringe assembly. In some embodiments, the shock absorber system does not comprise a shock absorber modifier; in other embodiments, the shock absorber includes a shock absorber modifier.

In some embodiments, upon release, the firing spring releases energy of at least about 5 lbs·in. In some embodiments, upon release the firing spring releases energy of at least about 8.7 lbs·in to about 12.3 lbs·in or more. In some embodiments, upon release the firing spring releases energy of at least about 8 lbs·in to about 10 lbs·in. In some embodiments, upon release the firing spring releases energy of at least about 9 lbs·in to about 12 lbs·in. In some embodiments, upon release the firing spring releases energy of at least about 5 lbs·in, about 6 lbs·in, about 7 lbs·in, about 7. 5 lbs·in, about 8 lbs·in, about 8.7 lbs·in, a about 9.2 lbs·in, about 10 lbs·in, about 11 lbs·in, about 12 lbs·in, about 12.3 lbs·in, about 13 lbs, about 14 lbs·in, about 15 lbs·in, about 16 lbs·in about 17 lbs in, about 18 lbs·in, about 19 lbs·in, about 20 lbs·in, about 25 lbs·in, about 30 lbs·in, about 45 lbs·in or about 60 lbs·in. In some embodiments, upon release the firing spring releases energy in the range of about 5 to about 60 lbs·in. In some embodiments, upon release the firing spring releases energy in the range of about 5 to about 30 lbs·in. In some embodiments, upon release the firing spring releases energy in the range of about 5 to about 20 lbs·in.

The invention further provides a single- or multi-use injector, comprising: (a) a housing; (b) a firing spring within the housing; (c) a syringe assembly in front of the firing spring; (d) a shock absorber system, wherein the shock absorber system comprises a stationary shock absorber; (e) a trigger mechanism, wherein the trigger mechanism is adapted to hold the firing spring in a cocked position until a user fires the automatic injector; and (f) a means for removing the syringe assembly from the housing. In some embodiments, the means for removing the syringe assembly from the housing is a screw threaded nose cap on the front of the housing. In some embodiments, the nose cap is threaded such that, after the automatic injector has been fired, twisting the nose cap about its axis causes the firing spring to become unloaded or substantially unloaded before the nose cap comes free from the automatic injector body. In some embodiments, the shock absorber is located within the nose cap. In some embodiments, the shock absorber system may also include a shock absorber, a shock absorber modifier or both on the syringe assembly. In some embodiments, upon release the firing spring releases energy of at least about 5 lbs·in. In some embodiments, upon release the firing spring releases energy of at least about 7 lbs·in. In some embodiments, upon release the firing spring releases energy of at least about 8 lbs·in. In some embodiments, upon release the firing spring releases energy of at least about 8.7 lbs·in. In some embodiments, upon release the firing spring releases energy of at least about 9 lbs·in. In some embodiments, upon release the firing spring releases energy of at least about 9.2 lbs·in. In some embodiments, upon release the firing spring releases energy of at least about 12.3 lbs·in. In some embodiments, upon release the firing spring releases energy in the range of about 5 to about 60 lbs·in. In some embodiments, upon release the firing spring releases energy in the range of about 5 to about 45 lbs·in. In some embodiments, upon release the firing spring releases energy in the range of about 5 to about 30 lbs·in. In some embodiments, the injector is adapted to deliver an automatic injection and at least one manual injection. In some embodiments, the injector is adapted to deliver a first automatic injection and a second manual injection. In some embodiments, the injector is adapted to deliver a first manual injection and a second automatic injection. In some embodiments, the injector is adapted to deliver two automatic injections and/or two manual injections. In some embodiments, the shock absorber system further includes a needle penetration controller located between the syringe assembly and the shock absorber. In some embodiments, the shock absorber system does not include a shock absorber on the syringe assembly. In some embodiments, the shock absorber system does not include a shock absorber modifier.

In further embodiments, the invention provides an automatic injector device comprising: (a) a housing; (b) a firing spring adapted to release greater than about 12 lbs·in of kinetic energy and located within the housing; (c) a syringe assembly in front of the firing spring; (d) a shock absorber capable of managing the kinetic energy imparted by said firing spring when the firing spring is released; and (e) a trigger mechanism, wherein the trigger mechanism is adapted to hold the firing spring in a cocked position until a user releases the firing spring, thereby firing the automatic injector. In some embodiments, the firing spring releases energy of at least about 15 lbs·in after it is released. In some embodiments, the firing spring releases energy of at least about 20 lbs·in after it is released. In some embodiments, the firing spring releases energy of at least about 25 lbs·in after it is released. In some embodiments, the firing spring releases energy of at least about 30 lbs·in after it is released. In some embodiments, the firing spring releases energy of at least about 45 lbs·in after it is released. In some embodiments, the firing spring releases energy in the range of about greater than about 12 lbs·in to about 60 lbs·in after it is released. In some embodiments, the shock absorber system does not include a shock absorber on the syringe assembly. In some embodiments, the shock absorber system does not include a shock absorber modifier assembly.

In further embodiments, the invention provides a method of reducing dynamic stresses on internal components of an automatic injector, comprising providing the automatic injector with a shock absorber system capable of managing energy released by a firing spring during firing, said energy released by the firing spring being at least about 8 lbs·in. In some embodiments, the energy released by the firing spring is at least about 9 lbs in. In some embodiments, the energy released by the firing spring is at least about 9.2 lbs·in. In some embodiments, the energy released by the firing spring is at least about 10 lbs·in. In some embodiments, the firing spring is at least about 11 lbs·in. In some embodiments, the energy released by the firing spring is at least about 12 lbs·in. In some embodiments, the energy released by the firing spring is at least about 12.3 lbs·in. In some embodiments, the energy released by the firing spring is at least about 20 lbs·in. In some embodiments, the energy released by the firing spring is in the range of about 8 to about 30 lbs·in. In some embodiments, the energy released by the firing spring is about 8 lbs·in, about 8.7 lbs·in, about 9 lbs·in, about 10 lbs·in, about 11 lbs·in, about 12 lbs·in, about 13 lbs·in, about 14 lbs·in, about 15 lbs·in, about 16 lbs·in about 17 lbs·in, about 18 lbs·in, about 19 lbs·in, about 20 lbs·in, about 21 lbs·in, about 22 lbs·in, about 23 lbs·in, about 24 lbs·in, about 25 lbs·in, about 26 lbs·in, about 27 lbs·in, about 28 lbs·in, about 29 lbs·in or about 30 lbs·in. In some embodiments, the shock absorber system comprises a stationary shock absorber. In some embodiments, the stationary shock absorber is located between a syringe assembly and a front end of the automatic injector. In some embodiments, the shock absorber system further comprises a needle penetration controller between the syringe assembly and the stationary shock absorber. In some embodiments, the automatic injector is a single- or multi-use automatic injector. In some embodiments, the automatic injector is a dual-use automatic injector. In some embodiments, the shock absorber system does not comprise a shock absorber on the syringe assembly; in other embodiments, the shock absorber system does include a shock absorber on the syringe assembly. In some embodiments, the shock absorber system does not comprise a shock absorber modifier; in other embodiments, the shock absorber includes a shock absorber modifier.

In some embodiments, the invention provides an automatic injector device comprising: (a) a housing; (b) a firing spring adapted to release less than about 8 lbs·in of kinetic energy and located within the housing; (c) a syringe assembly in front of the firing spring; (d) a shock absorber system capable of managing the kinetic energy imparted by said firing spring when the firing spring is released; and (e) a trigger mechanism, wherein the trigger mechanism is adapted to hold the firing spring in a cocked position until a user releases the firing spring, thereby firing the automatic injector. In some embodiments, the firing spring is adapted to release energy of about 7 lbs·in to less than about 8 lbs·in after it is released. In some embodiments, the firing spring is adapted to release energy of about 6 lbs·in to less than about 8 lbs·in after it is released. In some embodiments, the firing spring is adapted to release energy of about 5 lbs·in to less than about 8 lbs·in after it is released. In some embodiments, the firing spring is adapted to release energy about 4 lbs·in to less than about 8 lbs·in after it is released. In some embodiments, the shock absorber system does not comprise a shock absorber on the syringe assembly; in other embodiments, the shock absorber system does include a shock absorber on the syringe assembly. In some embodiments, the shock absorber system does not comprise a shock absorber modifier; in other embodiments, the shock absorber includes a shock absorber modifier. In some embodiments, the shock absorber system comprises a stationary shock absorber as the sole shock absorber.

In some embodiments, the invention provides an automatic injector device comprising: (a) a housing; (b) a firing spring adapted to release less than about 29 lbs·in of energy and located within the housing; (c) a syringe assembly in front of the firing spring; and (d) a trigger mechanism, wherein the trigger mechanism is adapted to hold the firing spring in a cocked position until a user releases the firing spring, thereby firing the automatic injector. In some embodiments, the firing spring is adapted to release less than about 27 lbs·in of energy. In some embodiments, the firing spring is adapted to release less than about 25 lbs·in of energy. In some embodiments, the firing spring is adapted to release less than about 20 lbs·in of energy. In some embodiments, the firing spring is adapted to release about 5 lbs·in, about 6 lbs·in, about 7 lbs·in, about 7.5 lbs·in, about 8 lbs in, about 8.7 lbs·in, about 9 lbs·in, about 10 lbs·in, about 11 lbs·in, about 12 lbs·in, about 13 lbs·in, about 14 lbs·in, about 15 lbs·in, about 16 lbs·in about 17 lbs·in, about 18 lbs·in, about 19 lbs·in, about 20 lbs·in, about 21 lbs·in, about 22 lbs·in, about 23 lbs·in, about 24 lbs·in, about 25 lbs·in, about 26 lbs·in, about 27 lbs·in or about 28 lbs·in. In some embodiments, the shock absorber system does not comprise a shock absorber on the syringe assembly; in other embodiments, the shock absorber system does include a shock absorber on the syringe assembly. In some embodiments, the shock absorber system does not comprise a shock absorber modifier; in other embodiments, the shock absorber includes a shock absorber modifier. In some embodiments, the injector is a single- or multi-use injector. In some embodiments, the injector is adapted to deliver a first automatic injection and a second manual injection, a first manual injection and a second automatic injection, two automatic injections or two manual injections.

In some embodiments, the invention provides an automatic injector device comprising: (a) a housing; (b) a firing spring adapted to release about 8 lbs·in to about 10 lbs·in of kinetic energy and located within the housing; (c) a syringe assembly in front of the firing spring; (d) a shock absorber capable of managing the kinetic energy imparted by said firing spring when the firing spring is released; and (e) a trigger mechanism, wherein the trigger mechanism is adapted to hold the firing spring in a cocked position until a user releases the firing spring, thereby firing the automatic injector. In some embodiments, the automatic injector is a single- or multi-use injector. In some embodiments, the automatic injector is adapted to deliver a first automatic injection and a second manual injection, a first manual injection and a second automatic injection, two automatic injections or two manual injections.

In some embodiments, the invention provides an automatic injector device comprising: (a) a housing; (b) a firing spring adapted to release about 8.7 lbs·in to about 12.3 lbs·in or more of energy and located within the housing; (c) a syringe assembly in front of the firing spring; (d) a shock absorber capable of managing the kinetic energy imparted by said firing spring when the firing spring is released; and (e) a trigger mechanism, wherein the trigger mechanism is adapted to hold the firing spring in a cocked position until a user releases the firing spring, thereby firing the automatic injector. In some embodiments, the automatic injector is a single- or multi-use injector. In some embodiments, the automatic injector is adapted to deliver a first automatic injection and a second manual injection, a first manual injection and a second automatic injection, two automatic injections or two manual injections.

In some embodiments, the invention provides an automatic injector device comprising: (a) a housing; (b) a firing spring adapted to release about 8.7 lbs·in to about 12.3 lbs·in of kinetic energy and located within the housing; (c) a syringe assembly in front of the firing spring; (d) a shock absorber capable of managing the kinetic energy imparted by said firing spring when the firing spring is released; and (e) a trigger mechanism, wherein the trigger mechanism is adapted to hold the firing spring in a cocked position until a user releases the firing spring, thereby firing the automatic injector. In some embodiments, the firing spring is adapted to release about 8 lbs·in, about 9 lbs·in, about 10 lbs·in, about 11 lbs·in or about 12 lbs·in of energy when released. In some embodiments, the automatic injector is a single- or multi-use injector. In some embodiments, the automatic injector is adapted to deliver a first automatic injection and a second manual injection, a first manual injection and a second automatic injection, two automatic injections or two manual injections.

In some embodiments, the invention provides an automatic injector device comprising a housing, a firing spring within the housing, a syringe assembly in front of the firing spring, a shock absorber system in front of the syringe assembly, and a trigger mechanism. The trigger mechanism is adapted to hold the firing spring in a cocked position until a user triggers release of the firing spring, thereby firing the automatic injector. The shock absorber system comprises a stationary shock absorber. In some specific embodiments, the shock absorber system may include at least one additional component, such as a needle penetration controller, an auxiliary shock absorber or an auxiliary shock absorber modifier. However, in preferred embodiments, the shock absorber system does not include a shock absorber on the syringe assembly (especially not on the needle hub). In some particular embodiments, the shock absorber system does not include a shock absorber modifier on the syringe assembly (especially not on the needle hub). In other particular embodiments, the shock absorber system includes neither a shock absorber nor a shock absorber modifier on the syringe assembly (especially not on the needle hub). In some embodiments, the automatic injector has a nose cap and the stationary shock absorber is located within the nose cap. In particular embodiments, the nose cap is threaded such that it can be unscrewed and thereby removed from the housing. In particular, the nose cap can be unscrewed by twisting the nose cap about an axis of the nose cap. In specific examples, the nose cap is adapted to move sufficient distance such that the firing spring becomes completely unloaded or substantially completely unloaded before the nose cap comes loose from the housing. This configuration has the advantage of allowing removal of the syringe assembly without the risk that the firing spring will launch the spring from the housing upon removal of the nose cap. In some embodiments, the nose cap has an inner shelf, which is an indentation on which the shock absorber rests. In some embodiments, the shock absorber system further comprises a needle penetration controller, which resides between the syringe assembly and the stationary shock absorber. In particular embodiments, the firing spring releases energy of at least about 5 lbs·in, at least about 7 lbs·in, at least about 7.5 lbs·in, at least about 8 lbs·in at least about 8.7 lbs·in, at least about 9 lbs·in, at least about 9.2 lbs·in, at least about 10 lbs·in, at least about 11 lbs·in, at least about 12 lbs·in or at least about 12.3 lbs·in. Particular ranges of energy released by the firing spring are about 5 to about 60 lbs·in, about 5 to about 50 lbs·in, about 5 to about 40 lbs·in, about 5 to about 30 lbs·in, about 5 to about 25 lbs·in, about 8 to about 10 lbs·in, about 9 to about 12 lbs·in, about 7 lbs·in, about 8 lbs·in, about 9 lbs·in, about 10 lbs·in, about 11 lbs·in, about 12 lbs·in, about 13 lbs·in, about 5 to or about 15 lbs·in, about 16 lbs·in about 17 lbs·in, about 18 lbs·in, about 19 lbs·in, about 20 lbs·in, about 21 lbs·in, about 22 lbs·in, about 23 lbs·in, about 24 lbs·in, about 25 lbs·in, about 26 lbs·in, about 27 lbs·in or about 28 lbs·in or greater.

In the context of the present invention, the term “about” means approximately and will be apparent to the person of skill in the art. For example, in some embodiments, “about” means within ±10% of the stated value. In some embodiments, the tolerance is about ±5% of the stated value.

In some embodiments, the invention provides a single- or multi-use injector, comprising a housing, a firing spring within the housing, a syringe assembly in front of the firing spring, a shock absorber system, a trigger mechanism, and a means for removing the syringe assembly from the housing after the automatic injector has been fired. In preferred embodiments, the shock absorber system comprises a stationary shock absorber. In some specific embodiments, the shock absorber system may include at least one additional component, such as a needle penetration controller, an auxiliary shock absorber or an auxiliary shock absorber modifier. However, in preferred embodiments, the shock absorber system does not include a shock absorber on the syringe assembly (especially not on the needle hub). In some particular embodiments, the shock absorber system does not include a shock absorber modifier on the syringe assembly (especially not on the needle hub). In other particular embodiments, the shock absorber system includes neither a shock absorber nor a shock absorber modifier on the syringe assembly (especially not on the needle hub). In preferred embodiments, the static shock absorber is located between the needle hub and the end of the housing, particularly within or substantially within the nose cone. The trigger mechanism is adapted to hold the firing spring in a cocked position until a user fires the automatic injector. The means for removing the syringe assembly from the housing after the automatic injector has been fired can be a screw-type nose cap, a separable housing or other means for allowing access to the syringe assembly. In some embodiments, the means for removing the syringe assembly from the housing is a screw threaded nose cap on the front of the housing. However, the nose cap may embody another type of mechanism for securing the nose cap to the housing, such as a bayonet, Luer or cam lock. In particular embodiments, the nose cap is threaded such that, after the automatic injector has been fired, twisting the nose cap about its axis causes the firing spring to become fully unloaded, or substantially fully unloaded, before the nose cap comes free from the automatic injector body.

As used herein, the term “substantially full unloaded” means that the spring is unloaded to a degree that a normal user may remove the nose cap without the syringe being forced out of the end of the housing at an unmanageable rate. In preferred embodiments, “substantially fully unloaded” means that the spring exerts no more than about 5 lbs of force, and in particular about 4 lbs or less, about 3 lbs or less or about 2 lbs or less of force when the nose cone has been fully unscrewed. In other preferred embodiments, the term “substantially fully unloaded” includes a final spring force of about 1 lbs or less. In particular embodiments, the firing spring is substantially unloaded, e.g. about 1 lb. of force or less remains in the firing spring, when the nose cone comes free from the injector body. In particular embodiments, the shock absorber is located within the nose cap, e.g. on a shelf or within an indentation in the inside of the nose cap. In some embodiments, the firing spring releases energy of at least about 4 lbs·in, about 5 lbs·in, at least about 6 lbs·in, at least about 7 lbs·in, at least about 8 lbs·in, at least about 8.7 lbs·in, at least about 9 lbs·in, at least about 10 lbs·in, at least about 11 lbs·in, or at least about 12 lbs·in. Particular ranges of energy released by the firing spring are about 5 lbs·in to about 60 lbs·in, about 5 lbs·in to about 50 lbs·in, about 5 lbs·in to about 40, about 5 lbs·in to about 30 lbs·in, about 5 lbs·in to about 25 lbs·in, about 8 to about 10 lbs·in, about 8.7 lbs·in to about 12.3 lbs·in, or about 9 to about 12 lbs·in. In some embodiments using a multi-use injector, the multi-use injector is capable of an automatic injection and at least one manual injection. In particular embodiments, the multi-use injector is capable of an automatic injection and one manual injection hi some such embodiments, the multi-use injector delivers the automatic injection as the first injection and the manual injection as the second injection. In other embodiments, the multi-use injector delivers the manual injection first and the automatic injection second. In still further embodiments, the multi-use injector delivers two automatic injections. In some embodiments, the shock absorber system further includes a needle penetration controller located between the syringe and the shock absorber.

In some embodiments, the invention provides an automatic injector device comprising a housing, a firing spring adapted to release greater than about 12 lbs·in of kinetic energy and located within the housing, a syringe assembly in front of the firing spring, a shock absorber capable of managing energy imparted by a released firing spring of greater than about 12 lbs·in, and a trigger mechanism. The trigger mechanism can be adapted to hold the firing spring in a cocked position until a user releases the firing spring, thereby firing the automatic injector. In some specific embodiments, the shock absorber system includes a stationary shock absorber. In more specific embodiments, the shock absorber system includes at least one additional component, such as a needle penetration controller, an auxiliary shock absorber or an auxiliary shock absorber modifier. However, in preferred embodiments, the shock absorber system does not include a shock absorber on the syringe assembly (especially not on the needle hub). In some particular embodiments, the shock absorber system does not include a shock absorber modifier on the syringe assembly (especially not on the needle hub). In other particular embodiments, the shock absorber system includes neither a shock absorber nor a shock absorber modifier on the syringe assembly (especially not on the needle hub). In some embodiments, the firing spring releases energy of at least about 12 lbs·in, at least about 13 lbs·in, at least about 20 lbs·in, at least about 25 lbs·in or 30 lbs·in or more after it is released; and the shock absorber is adapted to manage at least the amount of energy released by the firing spring. Particular ranges of energy released by the firing spring and managed by the shock absorber system are about 12 lbs·in to about 60 lbs·in, especially about 12 lbs·in to about 30 lbs·in, and in particular about 12 lbs·in, about 13 lbs·in, about 14 lbs·in, about 15 lbs·in, about 16 lbs·in about 17 lbs·in, about 18 lbs·in, about 19 lbs·in, about 20 lbs·in, about 21 lbs·in, about 22 lbs·in, about 23 lbs·in, about 24 lbs·in, about 25 lbs·in, about 26 lbs·in, about 27 lbs·in, about 28 lbs·in, about 29 lbs·in or about 30 lbs·in.

The invention also provides methods of reducing dynamic stresses on internal components of a single- or multi-use automatic injector. The methods comprise providing the automatic injector with a shock absorber capable of managing energy released by a firing spring during firing. In some embodiments, the energy released by the firing spring being at least about 5 lbs·in, at least about 10 lbs·in, at least about 15 lbs·in, at least about 20 lbs·in, at least about 25 lbs·in or at least about 30 lbs·in, at least about 45 lbs·in or about 60 lbs·in or greater. In some embodiments, the energy released by the firing spring is in the range of about 12 to about 30 lbs·in. In particular embodiments, the energy released by the firing spring is about 7.5 lbs·in, about 8 lbs·in, about 8.7 lbs·in, about 9 lbs·in, about 10 lbs·in, about 11 lbs·in, about 12 lbs·in, about 13 lbs·in, about 14 lbs·in, about 15 lbs·in, about 16 lbs·in about 17 lbs·in, about 18 lbs·in, about 19 lbs·in, about 20 lbs·in, about 21 lbs·in, about 22 lbs·in, about 23 lbs·in, about 24 lbs·in, about 25 lbs·in, about 26 lbs·in, about 27 lbs·in, about 28 lbs·in, about 29 lbs·in or about 30 lbs·in.

In particular embodiments, the shock absorber system comprises a stationary shock absorber. In specific embodiments the stationary shock absorber is located between a syringe assembly and a front end of the automatic injector. In some embodiments, the shock absorber system comprises a needle penetration controller between the syringe and the stationary shock absorber. In some embodiments, the automatic injector is a multi-use automatic injector, especially a dual-use automatic injector.

In some embodiments, the invention provides an automatic injector device comprising a housing, a firing spring adapted to release less than about 10 lbs·in of kinetic energy and located within the housing, a syringe assembly in front of the firing spring, a shock absorber capable of managing energy imparted by a released firing spring of less than about 10 lbs·in, and a trigger mechanism. In some embodiments, the shock absorber system comprises a stationary shock absorber. In some specific embodiments, the shock absorber system may include at least one additional component, such as a needle penetration controller, an auxiliary shock absorber or an auxiliary shock absorber modifier. However, in preferred embodiments, the shock absorber system does not include a shock absorber on the syringe assembly (especially not on the needle hub). In some particular embodiments, the shock absorber system does not include a shock absorber modifier on the syringe assembly (especially not on the needle hub). In other particular embodiments, the shock absorber system includes neither a shock absorber nor a shock absorber modifier on the syringe assembly (especially not on the needle hub). The trigger mechanism is adapted to hold the firing spring in a cocked position until a user releases the firing spring, thereby firing the automatic injector. In some embodiments, the firing spring releases energy of about 6 lbs·in to less than about 10 lbs·in, about 7 lbs·in to less than about 10 lbs·in, about 7.5 lbs·in to less than about 10 lbs·in, about 8 lbs·in to less than about 10 lbs·in, about 9 lbs·in to less than about 10 lbs·in after it is released.

In some embodiments, the invention provides an automatic injector device comprising a housing, a firing spring adapted to release about 8 lbs·in to about 10 lbs·in of kinetic energy and located within the housing, a syringe assembly in front of the firing spring, a shock absorber capable of managing energy imparted by a released firing spring of about 8 lbs·in to about 10 lbs·in and a trigger mechanism. In some embodiments, the shock absorber system comprises a stationary shock absorber. In some specific embodiments, the shock absorber system may include at least one additional component, such as a needle penetration controller, an auxiliary shock absorber or an auxiliary shock absorber modifier. However, in preferred embodiments, the shock absorber system does not include a shock absorber on the syringe assembly (especially not on the needle hub). In some particular embodiments, the shock absorber system does not include a shock absorber modifier on the syringe assembly (especially not on the needle hub). In other particular embodiments, the shock absorber system includes neither a shock absorber nor a shock absorber modifier on the syringe assembly (especially not on the needle hub). The trigger mechanism is adapted to hold the firing spring in a cocked position until a user releases the firing spring, thereby firing the automatic injector. In some embodiments, the device may be used to deliver two manual doses; however in preferred embodiments, the device is adapted to provide at least one automatically delivered dose. In more particular embodiments, the device is adapted to provide at least one automatic does and one manual dose or two automatic doses. In other particular embodiments, the device is adapted to provide a single, automatic, dose.

In some embodiments, the invention provides an automatic injector device comprising a housing, a firing spring adapted to release about 9 lbs·in to about 12 lbs·in of kinetic energy and located within the housing, a syringe assembly in front of the firing spring, a shock absorber capable of managing energy imparted by a released firing spring of about 9 lbs·in to about 12 lbs·in and a trigger mechanism. In some embodiments, the shock absorber system comprises a stationary shock absorber. In some specific embodiments, the shock absorber system may include at least one additional component, such as a needle penetration controller, an auxiliary shock absorber or an auxiliary shock absorber modifier. However, in preferred embodiments, the shock absorber system does not include a shock absorber on the syringe assembly (especially not on the needle hub). In some particular embodiments, the shock absorber system does not include a shock absorber modifier on the syringe assembly (especially not on the needle hub). In other particular embodiments, the shock absorber system includes neither a shock absorber nor a shock absorber modifier on the syringe assembly (especially not on the needle hub). The trigger mechanism is adapted to hold the firing spring in a cocked position until a user releases the firing spring, thereby firing the automatic injector. In some embodiments, the device may be used to deliver two manual doses; however in preferred embodiments, the device is adapted to provide at least one automatically delivered dose. In more particular embodiments, the device is adapted to provide at least one automatic does and one manual dose or two automatic doses. In other particular embodiments, the device is adapted to provide a single, automatic, dose.

In some embodiments, the invention provides an automatic injector device comprising a housing, a firing spring adapted to release less than about 29 lbs·in of energy and located within the housing, a syringe assembly in front of the firing spring, and a trigger mechanism. The trigger mechanism is adapted to hold the firing spring in a cocked position until a user releases the firing spring, thereby firing the automatic injector. In some embodiments, such an injector is a multi-use injector, and especially a dual-use injector. In some embodiments, the firing spring is adapted to release less than about 25 lbs·in, less than about 20 lbs·in, less than about 15 lbs·in or less than about 10 lbs·in of energy. In some embodiments, the firing spring is adapted to release about 5 lbs·in, about 6 lbs·in, about 7 lbs·in, about 7.5 lbs·in, about 8 lbs·in, about 8.7 lbs·in, about 9 lbs·in, about 9.2 lbs·in, about 10 lbs·in, about 11 lbs·in, about 12 lbs·in, about 12.3 lbs·in, about 13 lbs·in, about 14 lbs·in, about 15 lbs·in, about 16 lbs·in about 17 lbs·in, about 18 lbs·in, about 19 lbs·in, about 20 lbs·in, about 21 lbs·in, about 22 lbs·in, about 23 lbs·in, about 24 lbs·in, about 25 lbs·in, about 26 lbs·in, about 27 lbs·in or about 28 lbs·in.

In some embodiments, the device lacks a shock absorber system. In such embodiments, the released energy is managed by selecting a firing spring of suitable strength. In other embodiments, the automatic injector further comprises a shock absorber system. In some such embodiments, the shock absorber system comprises a stationary shock absorber. In particular embodiments, the shock absorber system, when present, does not include one or both of a shock absorber and/or a shock absorber modifier on the syringe assembly, and especially not on the needle hub.

In some embodiments, the present invention provides an improved shock absorber system for managing the energy delivered by a firing mechanism during operation of an automatic injector. The term “shock absorber system” means a system comprising at least one shock absorber. The shock absorber system may comprise additional components, such as a needle penetration controller; but as used herein the term “shock absorber system” requires the presence of at least one shock absorber. In some embodiments, the term “shock absorber” preferably includes an elastic disk or an elastic hollow cylinder (although other shapes or configurations may be utilized) that absorbs energy from one or more internal components of an automatic injector during firing. The improved shock absorber system of the present invention manages dynamic forces imparted to internal components during firing of the automatic injector. By better managing dynamic forces within the automatic injector during firing, the improved shock absorber reduces dynamic stresses on internal components and improves performance of the automatic injector. Further, by better managing dynamic forces within the automatic injector during firing, the improved shock absorber system permits use of firing mechanisms capable of releasing greater energy during firing, thereby enhancing the automatic injector's range of operating conditions. In some embodiments, the improved shock absorber system also provides advantages in the manufacture of the automatic injector, in that it is simpler to assemble. Other features and advantages will become apparent to the person of skill in the art as the improved energy management system and improved automatic injector are described in detail below.

In some embodiments, the invention provides an automatic injector that includes a shock absorber system that includes a stationary shock absorber. In contrast to the shock absorber system discussed above with respect to the prior art device, the stationary shock absorber according to the invention does not rest on the syringe assembly and does not travel with the syringe assembly during firing. In some embodiments, this reduces the total mass that the firing mechanism must push during firing, thereby enhancing the injector's performance. In addition, because the stationary shock absorber does not have to travel with the syringe, the stationary shock absorber may be more massive than the prior art shock absorber, thereby enhancing its dynamic energy management capacity as compared to the prior art shock absorber. Also, in some embodiments, employment of a stationary shock absorber reduces the complexity of the shock absorber system by eliminating the need for a shock absorber modifier and/or by eliminating the need during manufacturing to place the shock absorber over the end of the needle and onto the hub during assembly of the automatic injector. In some embodiments the stationary shock absorber is located toward the front end of the automatic injector, in particular between the needle hub and the front end of the automatic injector. In particular embodiments, the shock absorber system includes a needle penetration controller, which can be located between the needle hub and the stationary shock absorber.

FIGS. 4-6 show an automatic injector 200 according to certain embodiments of the invention. As depicted, the automatic injector 200 is a dual-use injector, meaning that it is adapted to deliver a first dose automatically (delivery of the medicament with the assistance of a spring) and a second dose manually. The person skilled in the art will appreciate that other embodiments are embraced by the present invention. Such modifications are described in more detail herein or are within the skill of the person skilled in the art. For purposes of describing the invention, all injectors capable of delivering at least one dose automatically are referred to herein as automatic injectors. Included in the meaning of the term “automatic injector,” as used herein, are injectors that are triggered manually (e.g. by depression of a button or trigger), but use a spring that releases stored energy to assist in delivery of at least one dose of medicine to a patient. Where it is desired to emphasize that the automatic injector is also capable of delivering at least one manual dose, the automatic injector may be referred to as a multiple use injector (or multi-use injector or multi-use automatic injector). Where the multiple dose injector is capable of delivering only two doses, one automatic and one manual, it is referred to herein as a dual-use injector (or dual-use automatic injector).

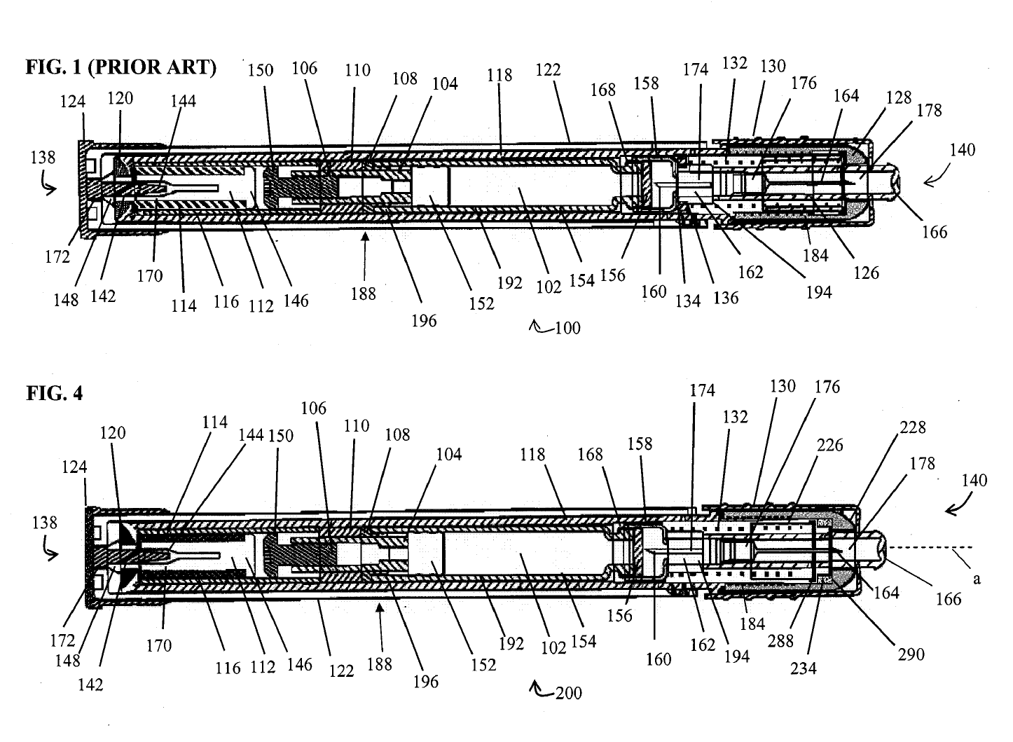

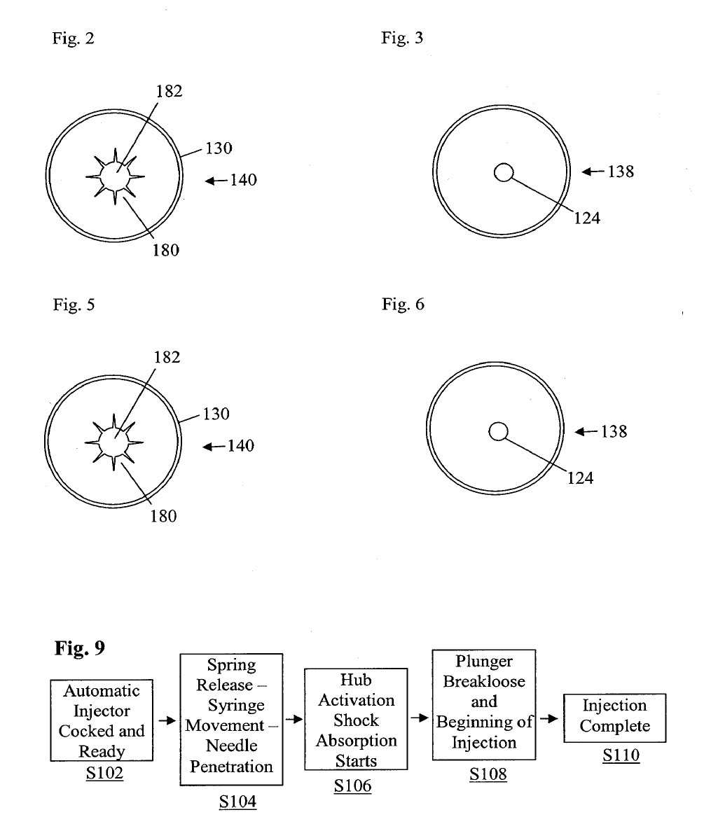

For purposes of convenience, the numbering used in FIGS. 4-6 duplicates the numbering used in FIGS. 1-3, except where it is necessary to distinguish parts of the improved automatic injector that differ from the prior art automatic injector. Moreover, the way in which the device in FIGS. 4-6 is fired is similar to that of the device in FIGS. 1-3. Some of the different and superior features of the automatic injector 200 are discussed below.

FIGS. 4-6 depict an automatic injector 200 according to the invention, wherein the shock absorber 234 is located between the syringe assembly 192 and the end of the barrel 118. In the embodiment depicted, the shock absorber is located within the nose cap 228. The improved shock absorber 234 is stationary in that it does not move with the syringe assembly 192 during firing. Thus, it may be made more massive than the prior art shock absorber 134. In particular, the person skilled in the art will recognize that the improved shock absorber 234 may be made larger in any dimension-longitudinally, radially and/or in aspect (the difference between its inner and outer diameter)-as compared to the prior art shock absorber. Thus the improved shock absorber 234 provides additional flexibility of automatic injector design as compared to that provided by the prior art shock absorber.

As depicted in FIG. 4, the automatic injector 200, does not employ a shock absorber modifier. This is in contrast to the prior art injector 100, which uses a shock absorber modifier 136 (FIG. 1) to spread force over the shock absorber 134. In the depicted embodiment, automatic injector 200, requires no such modifier for the shock absorber 234. Thus, in some embodiments, the invention provides a shock absorber system that excludes a separate shock absorber modifier. This leads to simpler, more efficient and less expensive assembly than is possible with the prior art injector, which is another advantage of certain embodiments of the improved shock absorber system over the prior art shock absorber system described above. Although it is preferred to use the stationary shock absorber without an auxiliary shock absorber (e.g. on the syringe assembly, and particularly on the needle hub) and/or a shock absorber modifier, in some embodiments the shock absorber system may include such additional components, as the resulting shock absorber system are envisioned as possessing at least the advantage of enhanced energy management capacity.

The automatic injector 200 of the invention comprises in certain embodiments, a barrel 118 and a firing sleeve 122, which together form housing 188. The barrel 118 fits within the lumen of the firing sleeve 122; and the firing sleeve 122 is capable of sliding without the barrel 118. For purposes of orientation, the automatic injector 200 can be envisioned as having a muzzle end 140 and a butt end 138. In the following description, the term “muzzle” may be used as a modifier indicating an orientation toward the muzzle end 140. This same direction may be referred to herein as the “front” or the “forward” end. The terms “butt,” “rear,” and “back,” may be used as a modifier indicating an orientation toward the butt end 138 of the automatic injector 200. Thus, a first component is said to be “in front” of a second component if the former is located more toward the muzzle end than the latter; and the latter would then be said to be “in back of” the former. Also, travel from the butt end toward the muzzle end of the barrel 118 may be referred to herein as being oriented “down” the barrel 118.

A trigger hole 142 in the butt end of the barrel 118 lines up with a release aperture 172 in the butt end of the firing sleeve 122, the operation of which is similar to that of the corresponding parts in prior art automatic injector I 00. A cylindrical spring guide 116 fits within the butt of the lumen of the barrel 118. Within the lumen of the spring guide 116 is a firing spring 114. A spring release 112 fits within the firing spring 114 in such a way as to hold the firing spring 114 in place in a cocked position. The spring release 112 has a spring release head 146 and a plurality of spring release legs 170 that terminate in spring release hooks 148. The firing spring 114 abuts the back of the spring release head 146. The firing spring 114 is compressed and spring release legs 170 protrude through the firing spring 114, the firing bushing 120 and the trigger hole 142. The spring release hooks 148 hold the spring release 112 in place. Thus the firing spring 114 is held in a compressed position until activated. The spring release hooks 148 are so shaped, and the trigger hole 142 and release aperture 172 are so sized, that when the firing sleeve 122 is moved toward the muzzle end 140 of the automatic injector 200 with sufficient force, the inner walls of the release aperture 172 push spring release hooks 148 inward until they are capable of passing through the trigger hole 142. This releases the firing spring 114, which is then free to impart stored energy to other internal components of the automatic injector 200, as discussed in more detail below.

With the firing spring 114 in the above-described cocked position, a safety cap 124, having a stem 144, fits on the butt end 138 of the firing sleeve 122. The stem 144 of the safety cap 124 fits through the release aperture 172, the spring release hooks 148, the trigger hole 142, the firing bushing 120 and the spring release legs 170. While the stem 144 is in place, it prevents inward motion of the spring release hooks 148, thereby preventing release of the cocked firing spring 114. Once the safety cap 124 is removed, however, the spring release hooks 148 are free to move inward. Forward motion of the firing sleeve 122 relative to the barrel 118 will then cause the inner wall of the release aperture 172 to press in on the outer edges of the spring release hooks 148, pushing them inward until they are free to pass through the trigger hole 142, thereby releasing the firing spring 114. Once released, the firing spring 114 is then free to push the spring release 112 in the direction of the muzzle end 140.

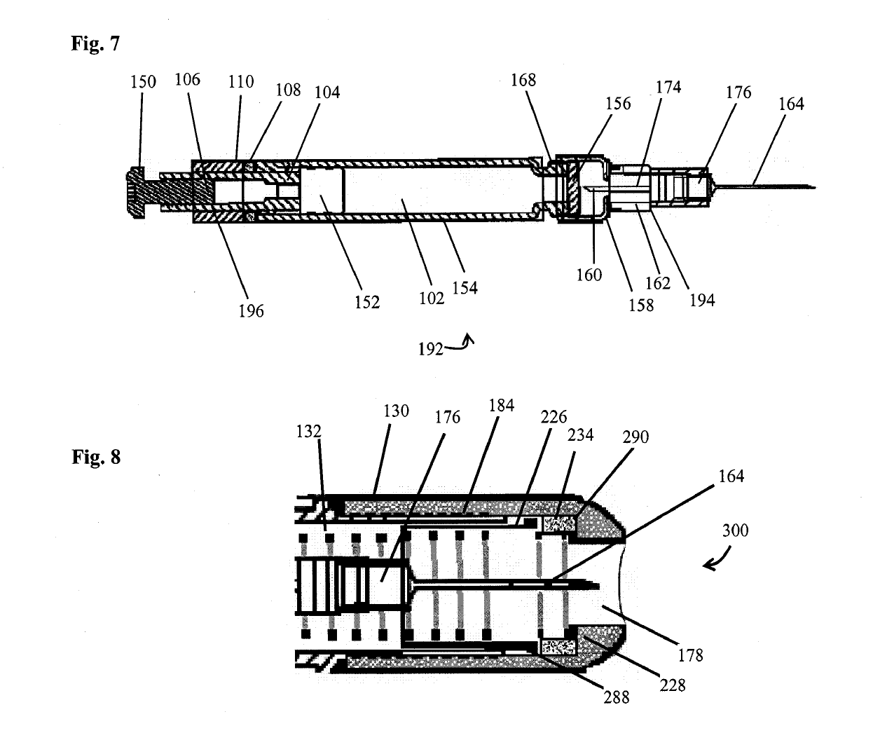

The automatic injector 200 in certain embodiments also has a syringe assembly 192, which comprises a syringe body 154, a plunger subassembly 196 and a needle hub subassembly 194. This syringe assembly 192 is essentially the same as that depicted in FIG. 7, and is described in detail above.

The barrel 118 has a removable, threaded nose cap 228 fitted over the muzzle (front) thereof. Within the nose cap 228 there is a cylindrical needle penetration controller 226 and a cylindrical shock absorber 234. The cylindrical needle penetration controller 226 is a relatively hard cylinder of appropriate material, such as a polymer material. The shock absorber 234 is an cylinder of elastic material, such as an elastic polymer material. The nose cap 228 has an inner shelf 290 on which the shock absorber 234 rests between the shelf 290 and the needle penetration controller 226. It is noted that, because the shock absorber 234 is not required to move with the syringe assembly 192 it can be taller (greater longitudinal dimension), thicker (greater aspect ratio), and of greater diameter (greater radial dimension) than was possible with the prior art shock absorber 134. Thus, the improved shock absorber 234 can absorb and disperse greater force than is possible with the prior art shock absorber 134. The person skilled in the art will recognize that, although the needle penetration controller 226 and shock absorber 234 are depicted as having certain apparent relative dimensions, their actual dimensions, both absolute and relative, may be varied within the scope of the present invention.

The person skilled in the art will recognize that the nose cap 228 may, in some embodiments, be non-removable, such that the automatic injector is adapted for administering a single, automatic dose only. For example, where manual use is not desired, the nose cap may be cemented onto the end of the barrel 118. The person skilled in the art will recognize that there are other, equivalent means to manufacture a non-removable nose cone, e.g. by manufacturing the nose cone 228 and the barrel 118 as a single, integrated unit. However, in the depicted embodiment, the nose cap 228 is threaded with threads 184 so that it may be removed by twisting it about an axis a running down the center of the nose cap 228.

The needle penetration controller 226 is a cylindrical structure that contacts the shock absorber 234 within the nose cap 228. Upon firing, the syringe assembly 192 moves down the barrel 118 (forward) until the hub 158 impacts the back of the needle penetration controller 226, which communicates the shock of the impact into the shock absorber 234, which smoothly stops the forward motion of the needle penetration controller 226 and consequently of the syringe assembly 192. As the syringe assembly 192 stops moving forward, the cap 168 within the hub 158 is pressed forward and is seated within the hub 158, while the engagement needle 160 pierces the septum 156 through the hole in the cap 168. The combination of seating of the cap 168 within the hub 158 and piercing of the septum 156 by the engagement needle 160 may also be referred to as “hub activation.” Through hub activation, the engagement needle 160 comes into contact with the medicament 102. The force of the firing spring 114 acting through the adjustment screw 150 of the plunger assembly 196 then begins to move the plunger 152 forward. This beginning of plunger movement is also referred to as plunger “break loose.” As the plunger 152 moves forward it pushes medicament 102 through the engagement needle 160, through and out of the injection needle 164 and into the patient.

Thus, the improved shock absorber system in certain embodiments of the invention can be envisioned as having a single piece, stationary shock absorber 234 located between the syringe assembly 192 and the end of the barrel 118. In some embodiments, such as the one depicted, the improved shock absorber system includes a needle penetration controller 226, removable nose cap 228 and the shock absorber 234. In other embodiments, the syringe may contact the stationary shock absorber directly, i.e. without an intervening nose cap.

As mentioned above, in some embodiments the nose cap need 228 need not be removable. In other embodiments, including the one depicted the nose cap 228 is removable by twisting it about its longitudinal axis a, thereby unscrewing it from the end of the barrel 118. This allows the user to remove the nose cap 228 and access the syringe assembly 192 for delivery of a second, manual, dose.

In some embodiments, the firing spring 114 may remain slightly compressed after firing, with the result that some potential energy remains stored in the firing spring 114. In such embodiments, it is advantageous for the nose cap 228 to be threaded with threads 184 such that the firing spring 114 becomes completely decompressed (unloaded) before the nose cap 228 reaches the end of its threading and completely detaches from the barrel 118. The person of skill in the art will recognize that this may be accomplished by using various combinations of steepness and number of turns of the screw threads 184 in the nose cap 228 such that the partially depressed firing spring 114 will become completely decompressed before the nose cap 228 can be removed from the barrel 118 by the user.

In the embodiment depicted in FIG. 4, a return spring 232 extends from the hub 158 to the inside of the shelf 290 within the nose cap 228. One function of the return spring 232 is to bias the syringe assembly 192 backward prior to firing. In general, the spring rate of the return spring 232 is much lower than that of the firing spring 114; and the return spring 232 thus provides very little damping force in opposition to the firing spring 114 during firing. Thus, as used herein, the terms “return spring” and “shock absorber” are distinct, the former referring to a spring extending from approximately the muzzle end of the barrel 118 to the syringe assembly 192 and the latter referring to a ring- or tube-shaped piece of elastic fitting between the muzzle end of the barrel 118 and the syringe assembly 192.

In the embodiment depicted, a needle sheath 166 fits through the needle aperture 178 and over the end of injection needle 164 to protect the injection needle 164 from damage and to protect the user and others from accidental needle sticks. A sheath remover 130 fits over the end of the nose cap 228 and has sheath receiving orifice 182 (FIG. 5), which is encircled by a plurality of projections 180 (FIG. 5). The projections 180 engage the needle sheath 166 and allow the sheath 166 to be easily removed from the injection needle 164 with reduced risk of an accidental needle stick to the user.

FIG. 8 shows an expanded cutaway view of an improved shock absorber system 300 of the invention. In the depicted embodiment, the shock absorber system 300 comprises the nose cap 228 having an inner shelf 290, a shock absorber 234 resting on the inner shelf 290 and a needle penetration controller 226, which has a cylindrical flange 288 at the end of the needle penetration controller 226 facing the shock absorber 234. In the depicted embodiment, the flange 288 holds the needle penetration controller 226 in place prior to firing by engaging the inside wall of the nose cone 228. Again it is noted that the relative dimensions of the shock absorber 234 and needle penetration controller 226 may vary greatly from those depicted in the drawings so long as a stationary shock absorber 234 remains between the inner shelf 290 and the syringe.

Also depicted in FIG. 8 are parts of the injection needle 164 and the hub nose 176, as well as the return spring 132. Again, as the shock absorber 234 of the invention is not required to move with the needle hub 194 during firing in preferred embodiments, it can be made larger in any dimension, and thus of greater damping capacity, than the prior art shock absorber. Also, although operation of the depicted device would not generally be impeded by inclusion of a shock absorber modifier between the needle penetration controller 226 and the needle hub 294 (or for that matter between the needle penetration controller 226 and the shock absorber 234), it is an advantage of the invention that no such modifier is necessary for proper operation of the shock absorber system 300 of the invention.

Thus, an automatic injector device of the invention in certain embodiments comprises a housing, a firing spring within the housing, a syringe assembly containing the medicament adjacent the firing spring, and a shock absorber adapted to absorb excess dynamic energy that is left over after the medicament has been injected into the patient. In certain embodiments of the invention, the shock absorber is located within a removable nose cone that fits over the end of the housing. In particular embodiments, the shock absorber is located on a shelf within the removable nose cone.

Thus, the invention provides an improved shock absorber system for an automatic injector, including a dual-use or multi-use automatic injector, as described herein. The improved shock absorber provides exceptional energy management, reducing dynamic stresses on internal components of the automatic injector during firing. In particular, the shock absorber system includes a stationary shock absorber, especially a stationary shock absorber located between the syringe and the muzzle end of the automatic injector. Moreover, in using a stationary shock absorber, the improved shock absorber system does not require, and in particular embodiments does not employ, a shock absorber that is located on or that travels with the syringe during firing of the automatic injector. Additionally, the improved shock absorber system does not require, and in particular embodiments specifically does not include a shock absorber modifier. More particularly, the improved shock absorber system does not require, and in specific embodiments does not include, a shock absorber modifier that is located on or that moves with the syringe during firing of the automatic injector. Thus, specific embodiments of the invention provide an automatic injector having a stationary shock absorber, but excluding a shock absorber, a shock absorber modifier or both a shock absorber and a shock absorber modifier that are on the syringe or travel with the syringe during firing of the automatic injector.

A firing spring according to the invention includes a spring designed to deliver the necessary force to move the syringe assembly (including the needle) down the barrel, push the needle into the patient, activate the hub and deliver the medicament to the patient through the needle.

In order to perform all the necessary functions during firing of the automatic injector, i.e. moving the syringe down the injector barrel, inserting the needle into the patient, activating the hub and injecting the medicament into the patient, the firing spring must, when compressed, be adapted to release sufficient energy to complete each of these actions. In general, it is advantageous to provide a spring capable of delivering excess energy in order for the device to operate under a range of operational conditions. Provision of excess potential energy in the spring ensures that, at each step in firing of the automatic injector, there will be adequate dynamic spring force to carry out that step throughout a range of ambient conditions. By providing enhanced management of dynamic forces within the automatic injector during firing, the improved shock absorber according to the invention supports use of springs capable of releasing greater amounts of energy than were supported by the prior art shock absorber. Thus, the improved shock absorber of the invention reduces stresses on internal components of the automatic injector during firing.

The spring rate (K) of a spring is the amount static spring force (expressed in 1 lb·f or lbs) per unit length of compression (measured in inches). Thus, the spring rate (K) is expressed in lbs/in. In some embodiments of the invention, the automatic injector comprises a spring having a spring rate (K) in the range of about 6 to about 30 lbs/in, e.g. in the range of about 7.0 to about 20 lbs/in, especially in the range of about 7.5 to about 15 lbs/in, especially about 5, about 6, about 7, about 8, about 9, about 10, about 11 or about 12 lbs/in.

The length of the firing spring may be varied within a range convenient for use in a device to be carried in a purse, backpack or pocket. In general, the length of the spring should be in the range of about 1 to about 10 in, especially from about 2 to about 5 in, and more particularly from about 2.5 to about 4 in. In some embodiments, spring lengths of about 2, about 2.5, about 3, about 3.5, about 4, about 4.5, about 5, about 5.5, about 6, about 6.5, about 7 or more inches may be used in practicing the invention. The diameter of the firing spring may also be varied. In some embodiments, the spring has a diameter of about 0.1, about 0.15, about 0.2, about 0.25, about 0.3, about 0.35, about 0.4, about 0.45 or about 0.5 in or more. In a particularly preferred embodiment, the spring has a length of 2.4 in (±10%) and a diameter of 0.3 in (±5%).

The energy released by a spring that is initially compressed X₁ (in) and that decompresses from X₁ in to X₂ (in), after it is released, is represented by the following formula (I):
ΔE=½K·(X₁²−X₂²), where (I)

wherein ΔE is the energy released (lbs·in), K is the spring constant (lbs/in), X₁ is the number of inches the spring is initially compressed (in) and X₂ is the number of inches the spring is compressed after it has been released (in). Note that this relationship can be generalized, such that ΔE is the energy released when the spring decompresses from any X₁ to any X₂. This relationship is also independent of units chosen; i.e. instead of inches, centimeters, meters, feet or some other unit of length may be chosen.

When the firing spring 114 is released, it exerts a force on the syringe assembly 192 and moves the syringe assembly 192 down the lumen of the barrel 118 toward the muzzle end 140 of the automatic injector 200. Thus potential energy initially stored in the cocked firing spring 114 is imparted to the syringe assembly 192 and is further used by the device to insert the needle 116 into the patient, activate the hub 158 and injecting the medicament 102 into the patient. The amount of energy released by the firing spring 114 is governed by formula (I), above.

The shock absorber according to the invention is able to manage large amounts of energy released by a firing spring, thereby reducing dynamic stresses on internal components of the automatic injector. The energy released by the firing spring during firing may be referred to herein as “kinetic energy.” In some embodiments, the firing spring releases at least about 5 lbs·in of kinetic energy, especially at least about 7 lbs·in, at least about 7.5 lbs·in, at least about 8 lbs·in, at least about 8.7 lbs·in, at least about 9 lbs·in, at leas about 9.2 lbs·in, at least about 10 lbs·in, at least about 11 lbs·in, at least about 12 lbs·in, at least about 12.3 lbs·in or greater. In some embodiments, the shock absorber of the invention is capable of managing kinetic energies of greater than about 15, greater than about 25, greater than about 30 and/or greater than about 45 lbs·in or about 60 lbs·in or greater. The improved shock absorber is able to manage such kinetic energy, thereby reducing dynamic stresses on internal components of the automatic injector. The shock absorber is thus capable of managing, in some embodiments, kinetic energy in the range of about 5 to about 60 lbs·in, especially about 5 to about 45 lbs·in and more particularly about 5 to about 30 lbs·in. By reducing dynamic stresses on internal components of the automatic injector, the shock improved shock absorber of the invention permits, in some cases, the use of firing springs capable of delivering larger amounts of energy.

In some embodiments, the invention provides an automatic injector comprising a housing, a firing spring within the housing, a syringe assembly in front of the firing spring, and a trigger mechanism capable of releasing the firing spring when actuated by a user. The energy released by the firing spring is selected so as. to manage the dynamic forces acting on internal components of the injection device, thereby reducing dynamic stresses suffered by internal components during firing. In some embodiments, the firing spring is adapted to release less than about 29 lbs·in of energy. In particular embodiments, the firing spring is adapted to release less than about 25 lbs·in, especially less than about 20 lbs·in, more especially less than about 15 lbs·in, and even more particularly less than about 10, less than about 9, less than about 8, less than about 7, less than about 6 or less than about 5 lbs·in. In particular embodiments, the injector has no shock absorber and/or no shock absorber modifier on the syringe; and in specific embodiments the injector has no shock absorber whatsoever, although a device having a stationary shock absorber is preferred for the reasons stated above, especially when the firing spring releases energies of 8 lbs·in or greater. The trigger mechanism is adapted to hold the firing spring in a cocked position until a user releases the firing spring, thereby firing the automatic injector.

Thus, in some embodiments, the invention provides an improved automatic injector comprising a housing, a firing spring, a syringe assembly in front of the firing spring, a shock absorber capable of managing energy of about 7.5 lbs·in to about 10.5 lbs·in, which is imparted by releasing the firing spring, and a trigger mechanism. In particular embodiments, the improved automatic injector comprises a stationary shock absorber. In some specific embodiments, the improved automatic injector has no shock absorber and/or no shock absorber modifier on the syringe. In other particular embodiments, the improved automatic injector comprises a shock absorber on the syringe. In specific embodiments, an improved automatic injector comprises a shock absorber and/or a shock absorber modifier on the syringe.

Thus, in some embodiments, the invention provides an improved automatic injector comprising a housing, a firing spring, a syringe assembly in front of the firing spring, a shock absorber capable of managing energy of about 8.7 lbs·in to about 12.3 lbs·in, which is imparted by releasing the firing spring, and a trigger mechanism. In particular embodiments, the improved automatic injector comprises a stationary shock absorber. In some specific embodiments, the improved automatic injector has no shock absorber and/or no shock absorber modifier on the syringe. In other particular embodiments, the improved automatic injector comprises a shock absorber on the syringe. In specific embodiments, an improved automatic injector comprises a shock absorber and/or a shock absorber modifier on the syringe.

Thus, in some embodiments, the invention provides an improved automatic injector comprising a housing, a firing spring, a syringe assembly in front of the firing spring, a shock absorber capable of managing energy of about 8 lbs·in to about 10 lbs·in, which is imparted by releasing the firing spring, and a trigger mechanism. In particular embodiments, the improved automatic injector comprises a stationary shock absorber. In some specific embodiments, the improved automatic injector has no shock absorber and/or no shock absorber modifier on the syringe. In other particular embodiments, the improved automatic injector comprises a shock absorber on the syringe. In specific embodiments, an improved automatic injector comprises a shock absorber and/or a shock absorber modifier on the syringe.

Thus, in some embodiments, the invention provides an improved automatic injector comprising a housing, a firing spring, a syringe assembly in front of the firing spring, a shock absorber capable of managing energy of about 9 lbs·in to about 12 lbs·in, which is imparted by releasing the firing spring, and a trigger mechanism. In particular embodiments, the improved automatic injector comprises a stationary shock absorber. In some specific embodiments, the improved automatic injector has no shock absorber and/or no shock absorber modifier on the syringe. In other particular embodiments, the improved automatic injector comprises a shock absorber on the syringe. In specific embodiments, an improved automatic injector comprises a shock absorber and/or a shock absorber modifier on the syringe.

In other embodiments, the invention provides an improved automatic injector comprising a housing, a firing spring, a syringe assembly in front of the firing spring, a shock absorber capable of managing energy of less than about 8 lbs·in to about 10 lbs·in, which is imparted by releasing a firing spring, and a trigger mechanism. In particular embodiments, the improved automatic injector comprises a stationary shock absorber. In some specific embodiments, the improved automatic injector has no shock absorber and/or no shock absorber modifier on the syringe. In other particular embodiments, the improved automatic injector comprises a shock absorber on the syringe. In specific embodiments, an improved automatic injector comprises a shock absorber and/or a shock absorber modifier on the syringe.

In further embodiments, the invention provides an improved automatic injector comprising a housing, a firing spring, a syringe assembly in front of the firing spring, a shock absorber capable of managing energy of less than about 9 lbs·in to about 12 lbs·in, which is imparted by releasing a firing spring, and a trigger mechanism. In particular embodiments, the improved automatic injector comprises a stationary shock absorber. In some specific embodiments, the improved automatic injector has no shock absorber and/or no shock absorber modifier on the syringe. In other particular embodiments, the improved automatic injector comprises a shock absorber on the syringe. In specific embodiments, an improved automatic injector comprises a shock absorber and/or a shock absorber modifier on the syringe.

In some embodiments, the injector of the invention is operable within reasonable tolerances within a range of about 0° C. to about 55° C., especially in the range of about 5° C. to about 40° C.

### EXAMPLE 1

The present invention is illustrated with reference to illustrative, non-limiting examples designed to demonstrate possible advantages of automatic injectors employing an embodiment of a shock absorber system of the present invention as compared to a prior art automatic injector using a prior art shock absorber system, using a shock absorber on the syringe as the sole shock absorber. The static spring force in foot-pounds (1 lb·f or 1 lbs) is provided for the prior art injector and for an injector according to the present invention. For comparison, the prior art injector will use a shock absorber on the syringe and a firing spring having a spring coefficient (K) of 7.5 pounds per inch (lbs/in), whereas an embodiment of an injector according to the invention will use a stationary shock absorber in the nose of the injector as the sole shock absorber and a firing spring having a spring coefficient (K) of 11 lbs/in.

As discussed in more detail above, firing of an automatic injector requires that the firing spring provide adequate force for each step of firing. Table 1 below provides a comparison of the force available with a prior art injector employing the shock absorber system essentially as depicted in FIG. 1 with the aforementioned automatic injector according to an embodiment of the invention. Each of the points of reference corresponds to a step as depicted in the flow chart in FIG. 9, which shows specific steps in the operation of an automatic injector. The static spring forces recorded in Table 1 are the static spring forces calculated at the beginning of each step.

Specifically, in S102, the firing spring is in the cocked position and ready to fire. In S104, the firing sleeve is moved forward to release the firing spring; the firing spring pushes the spring release against the adjustment screw of the plunger subassembly. At this stage, the plunger has not yet started to move forward within the syringe barrel, so full force of the firing spring pushes the syringe down the lumen of the barrel. As the syringe assembly moves down the lumen of the barrel, the injection needle pierces and penetrates the skin of the patient. In S106, the needle hub impacts the needle penetration controller and shock absorption begins; the syringe cap becomes seated within the hub; and the activation needle pierces the septum such that the activation needle comes into contact with the medicament. In S108, the plunger begins to break loose, pushing medicament through the needle and into the patient. In S110, the medicament will be fully delivered.

TABLE 1

| | Static Spring | Static Spring |
|---|---|---|
| | Force | Force |
| | Prior Art Injector | Inventive Injector |
| Step | K = 7.5 lbs/in | K = 11 lbs/in |
| (See FIG. 9) | (lbs) | (lbs) |
| S102 | 13 | 17 |
| S106 | 8 | 11 |
| S108 | 7 | 10 |
| S110 | 4 | 7 |

As can be seen from Table 1, injectors according to embodiments of the present invention in this example will permit the use of a spring having a greater spring rate (11 lbs/in, respectively) than the prior art injector (K=7.5 lbs/in). Additionally, the injectors according to the present invention will also permit use of a spring having a higher total static spring force (17 lb·f) than the prior art injector (13 lb·f). (It is understood in the art that 1 lb·f=1 lbs of force). Additionally, injectors of the present invention will permit use of a spring having excess static spring force at the end of injection (S110) of as much as 7 lb·f, whereas the prior art will provide only about 4 lb·f of excess energy at the end of injection (S110). At each of the delineated steps, the injector according to the invention will provide increased static spring force, while managing the dynamic stresses imposed on the internal components of the device. Thus, in some embodiments, the injector of the invention provides additional static spring force, for operation under a variety of environmental conditions.

The static spring forces of Table 1 in this example can be translated into energies released during the various intervals after spring release. These intervals are summarized as S102-S106 (release to hub activation), S106-S108 (hub activation to plunger break loose and commencement of dose delivery) and S108-S110 (commencement of dose delivery to dose completion). For comparison the total energies that would be released by a prior art automatic injector (K=7.5 lbs/in), and two embodiments of injectors according to the invention (K=8.3 lbs/in and 11 lbs/in, respectively), are summarized in Table 2, below.

The prior art injector, having a shock absorber and shock absorber modifier on the syringe and having no stationary shock absorber, would have a spring rate (K) of 7.5 lbs/in. The first injector of the invention would have a spring rate (K) of 8.3 lbs/in, a shock absorber and a shock absorber modifier on the syringe and a stationary shock absorber in the nose of the injector. The second injector of the invention would have a spring rate (K) of 11 lbs/in and a stationary shock absorber in the nose of the injector. The energies are calculated using formula (I) above. ΔE is the energy that would be released between S102 and S110.

TABLE 2

| Device | K (lbs/in) | ΔE (lbs · in) |
|---|---|---|
| Prior Art (FIG. 1) | 7.5 | 8.8 |
| Invention (FIG. 4) | 8.3 | 9.1 |
| First Embodiment | | |
| Invention (FIG. 4) | 11 | 12 |
| Second | | |
| Embodiment | | |

As can be seen in Table 2 above, automatic injectors according to embodiments of the invention, which employ improved shock absorbers according to embodiments of the invention, can manage greater total energy (9.1 lbs·in and 12 lbs·in) than the prior art device (8.8 lbs·in). In fact, the exemplified embodiments of the improved shock absorber of the invention would manage increased energies and increased static spring forces at each interval of firing. By managing the dynamic stresses imposed upon internal components, embodiments of the automatic injector of the invention can take advantage of stronger springs and higher energies than can prior art automatic injectors without the shock absorber systems of the present invention, which employ stationary shock absorbers.

While preferred embodiments of the present invention have been described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An automatic injector device comprising:
(a) a housing;
(b) a firing spring within the housing;
(c) a syringe assembly in front of the firing spring;
(d) a shock absorber system in front of the syringe assembly, wherein the shock absorber system comprises a stationary shock absorber; and
(e) a trigger mechanism, wherein the trigger mechanism is adapted to hold the firing spring in a cocked position until a user triggers release of the firing spring, thereby firing the automatic injector.

2. The automatic injector of claim 1, wherein the housing has a nose cap and the stationary shock absorber is located within the nose cap.

3. The automatic injector of claim 2, wherein the nose cap is threaded and removable by twisting the nose cap about an axis of the nose cap.

4. The automatic injector of claim 3, wherein twisting the nose cap after the automatic injector has been fired causes the firing spring to become fully unloaded or substantially fully unloaded before the nose cap is removed.

5. The automatic injector of claim 2, wherein the nose cap has an inner shelf and the shock absorber rests at least in part on the inner shelf.

6. The automatic injector of claim 1, wherein the shock absorber system further comprises a needle penetration controller located between the syringe assembly and the shock absorber.

7. The automatic injector of claim 1, wherein the shock absorber system does not comprise a shock absorber on the syringe assembly.

8. The automatic injector of claim 1, wherein the shock absorber system includes a shock absorber on the syringe assembly.

9. The automatic injector of claim 8, wherein the shock absorber system further comprises a shock absorber modifier.

10. The automatic injector of claim 1, wherein upon release the firing spring releases energy of at least about 5 lbs·in.

11. The automatic injector of claim 1, wherein upon release the firing spring releases energy of at least about 8.7 lbs·in to about 12.3 lbs·in.

12. The automatic injector of claim 11, wherein upon release the firing spring releases energy of at least about 9 lbs·in to about 12 lbs·in.

13. A method of reducing dynamic stresses on internal components of an automatic injector, comprising providing the automatic injector with a shock absorber system capable of managing energy released by a firing spring during firing, said energy released by the firing spring being at least about 8.7 lbs in.

14. The method of claim 13, wherein the energy released by the firing spring is at least about 9 lbs·in.

15. The method of claim 13, wherein the energy released by the firing spring is at least about 10 lbs·in.

16. The method of claim 13, wherein the energy released by the firing spring is at least about 11 lbs·in.

17. The method of claim 13, wherein the energy released by the firing spring is at least about 12 lbs·in.

18. The method of claim 13, wherein the shock absorber system comprises a stationary shock absorber.

19. The method of claim 18, wherein the stationary shock absorber is located between a syringe assembly and a front end of the automatic injector.

20. The method of claim 13, wherein shock absorber system further comprises a needle penetration controller between the syringe assembly and the stationary shock absorber.

21. The method of claim 13, wherein the automatic injector is a multi-use automatic injector.

22. The method of claim 13, wherein the automatic injector is a dual-use automatic injector.

23. An automatic injector device comprising:
(a) a housing;
(b) a firing spring adapted to release about 8.7 lbs·in to about 12.3 lbs·in of energy and located within the housing;
(c) a syringe assembly in front of the firing spring;
(d) a shock absorber capable of managing the energy imparted by said firing spring when the firing spring is released; and
(e) a trigger mechanism, wherein the trigger mechanism is adapted to hold the firing spring in a cocked position until a user releases the firing spring, thereby firing the automatic injector.

24. An automatic injector of claim 23, wherein the automatic injector is a multi-use injector.

25. The automatic injector of claim 24, wherein the automatic injector is adapted to deliver a first automatic injection and a second manual injection, a first manual injection and a second automatic injection, two automatic injections or two manual injections.

26. An automatic injector device comprising:
(a) a housing;
(b) a firing spring adapted to release about 9 lbs·in to about 12 lbs·in of energy and located within the housing;
(c) a syringe assembly in front of the firing spring;
(d) a shock absorber capable of managing the energy imparted by said firing spring when the firing spring is released; and
(e) a trigger mechanism, wherein the trigger mechanism is adapted to hold the firing spring in a cocked position until a user releases the firing spring, thereby firing the automatic injector.

27. The automatic injector of claim 26, wherein the firing spring is adapted to release about 9 lbs·in, about 10 lbs·in, about 11 lbs·in or about 12 lbs·in of energy when released.

28. An automatic injector of claim 26, wherein the automatic injector is a multi-use injector.

29. The automatic injector of claim 26, wherein the automatic injector is adapted to deliver a first automatic injection and a second manual injection, a first manual injection and a second automatic injection, two automatic injections or two manual injections.
